(19) United States
(12) Patent Application Publication
(10) Pub. No.: US 2021/0059644 A1
(43) Pub. Date: Mar. 4, 2021

(54) RETROSPECTIVE MULTIMODAL HIGH FRAME RATE IMAGING

(71) Applicant: Shenzhen Mindray Bio-Medical Electronics Co., Ltd., Nanshan (CN)

(72) Inventors: Donglai Liu, San Jose, CA (US); Ting-lan Ji, Morgan Hill, CA (US); Glen W. McLaughlin, San Carlos, CA (US)

(21) Appl. No.: 16/814,466

(22) Filed: Mar. 10, 2020

Related U.S. Application Data
(60) Provisional application No. 62/834,547, filed on Apr. 16, 2019.

(51) Int. Cl. A61B 8/08; A61B 8/00

(52) U.S. Cl. CPC A61B 8/5284; A61B 8/488; A61B 8/5246; A61B 8/465

(57) ABSTRACT

Systems and methods for performing ultrasound imaging. Ultrasound information of a subject region in response to ultrasound pulses transmitted toward the subject region can be collected. One or more ultrasound images of at least a portion of the subject region can be formed using the ultrasound information. The ultrasound information can be stored in a memory. In turn, the ultrasound information can be accessed from the memory. Subsequently, the ultrasound information accessed from the memory can be reprocessed to retrospectively generate one or more additional ultrasound images of at least a portion of the subject region.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### PRIORITY APPLICATIONS

This application claims priority to U.S. Provisional Patent Application No. 62/834,547 to Donglai Liu et al., titled RETROSEPCTIVE MULTIMODAL HIGH FRAME RATE IMAGING, and filed Apr. 16, 2019, the entire disclosure of which is hereby incorporated herein by this reference.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### TECHNICAL FIELD

The present disclosure relates to ultrasound imaging and more particularly to storing collected ultrasound information in a memory and reprocessing the ultrasound information stored in the memory to retrospectively generate one or more additional ultrasound images.

### BACKGROUND OF THE INVENTION

Ultrasound imaging is widely used for examining a wide range of materials and objects across a wide array of different applications. Ultrasound imaging provides a fast and easy tool for analyzing materials and objects in a non-invasive manner. As a result, ultrasound imaging is especially common in the practice of medicine as an ailment diagnosis, treatment, and prevention tool. Specifically, because of its relatively non-invasive nature, low cost and fast response time ultrasound imaging is widely used throughout the medical industry to diagnose and prevent ailments. Further, as ultrasound imaging is based on non-ionizing radiation it does not carry the same risks as other diagnosis imaging tools, such as X-ray imaging or other types of imaging systems that use ionizing radiation.

Different imaging modes are used to investigate different aspects of physiology, such as tissue morphology, tissue motion, and blood flow. A limitation of current systems is that imaging modes, e.g. B-mode, color-flow mode, pulse-wave Doppler mode, tissue Doppler mode, tissue strain mode, tissue elasticity mode, and vector flow mode, are only selectable during live scanning, e.g. during a session. Specifically, once a session ends and a subject region is removed from an ultrasound imaging system, the collected ultrasound information is typically not stored for later use. In turn, this limits the ability of operators to retrospectively form images through different imaging modes using the collected ultrasound information. As follows, this limits the ability to conduct additional diagnosis using the collected ultrasound information after the session has ended.

### SUMMARY

According to various embodiments, a method for performing ultrasound imaging includes collecting ultrasound information of a subject region in response to ultrasound pulses transmitted toward the subject region. One or more ultrasound images of at least a portion of the subject region can be formed using the ultrasound information. Further, the ultrasound information can be stored in a memory. In turn, the ultrasound information can be accessed from the memory. As follows, the ultrasound information accessed from the memory can be reprocessed to retrospectively generate one or more additional ultrasound images of at least a portion of the subject region.

In certain embodiments, a system for performing ultrasound imaging includes an ultrasound transducer and a main processing console. The ultrasound transducer can collect ultrasound information of a subject region in response to ultrasound pulses transmitted toward the subject region. The main processing console can form one or more ultrasound images of at least a portion of the subject region using the ultrasound information. The main processing console can also store the ultrasound information in a memory. In turn, the main processing console can access the ultrasound information from the memory. As follows, the main processing console can reprocess the ultrasound information accessed from the memory to retrospectively generate one or more additional ultrasound images of at least a portion of the subject region.

In various embodiments, a system for performing ultrasound imaging includes one or more processors and a computer-readable medium providing instructions accessible to the one or more processors to cause the one or more processors to collect ultrasound information of a subject region in response to ultrasound pulses transmitted toward the subject region. The instructions can further cause the one or more processors to form one or more ultrasound images of at least a portion of the subject region using the ultrasound information. Additionally, the instructions can cause the one or more processors to store the ultrasound information in a memory. In turn, the instructions can cause the one or more processors to access the ultrasound information from the memory. As follows, the instructions can cause the one or more processors to reprocess the ultrasound information accessed from the memory to retrospectively generate one or more additional ultrasound images of at least a portion of the subject region.

### BRIEF DESCRIPTION OF THE DRAWINGS

FIG. 1 illustrates an example of an ultrasound system.

FIG. 2 is a flowchart of an example method for storing and reprocessing collected ultrasound information to generate additional ultrasound image(s) after the ultrasound information is collected.

FIG. 3 illustrates an example scan sequence of a high frame rate ultrasound imaging mode.

FIG. 4 illustrates another example scan sequence of a high frame rate ultrasound imaging mode.

FIG. 5 shows an example display format where the high frame rate image(s) are displayed in a separate region from a background B-mode image during a live imaging session.

FIG. 6 shows another example display format where the high frame rate image(s) are displayed embedded in the background B-mode image.

### DETAILED DESCRIPTION

Imaging in different ultrasound imaging modes can be used in identifying different characteristics of a subject region. As follows, the different imaging modes can be used investigate different aspects of physiology, such as tissue morphology, tissue motion and blood flow. In turn, the different imaging modes can allow doctors to more easily diagnose diseases and provide treatments for the diseases based on their diagnoses.

As discussed previously, current ultrasound imaging systems only allow for the application of specific imaging modes, and potentially different imaging modes, during an actual ultrasound session, e.g. while a subject region is being subjected to ultrasound transmit events. Specifically, current ultrasound imaging systems do not allow for the application of specific imaging modes, and potentially different imaging modes, after an ultrasound session has ended and the subject region is removed from the ultrasound imaging system. More specifically, current ultrasound imaging systems do not store collected ultrasound information after an ultrasound session to allow for the application of specific imaging modes, and potentially different imaging modes, after the ultrasound session has ended. As a result, this makes it difficult for doctors to easily diagnose diseases and retrospectively process ultrasound information after an ultrasound session has ended.

Further, with each transmit event in current ultrasound systems, the system's computational speed can only form a few (e.g. 1 to 8) receive beams in parallel. Therefore, to form a whole image made up of hundreds of beams, many transmit events are needed. In turn, this limits the frame rate capabilities of current ultrasound systems due to the time needed for sound to propagate. As follows, this can increase overall ultrasound session times.

The following disclosure describes systems, methods, and computer-readable media for solving these problems/discrepancies. Specifically, the present technology involves system, methods, and computer-readable media for storing ultrasound information gathered during an ultrasound session for reprocessing after the session. More specifically, the present technology involves systems, methods, and computer-readable media for forming one or more ultrasound images in a first imaging mode during an ultrasound session using ultrasound information gathered during the session. In turn, the ultrasound information is stored for retrieval and reprocessing after the session to generate one or more additional ultrasound images in a second imaging mode.

Specifically and as will be discussed in greater detail later, either collected channel domain data or image data of a subject region in a cyclical memory in an applicable format, e.g. in a RF data format for the channel domain data and an in-phase quadrature (IQ) data format for the image data. The ultrasound data can be stored in cyclical memory for a duration that can cover multiple cardiac cycles. Further, the stored ultrasound data can be reprocessed in different ways to apply specific, and potentially different, imaging modes retrospectively after an ultrasound session has ended, e.g., after a patient has left the medical office.

Additionally, a large amount, e.g. hundreds, of receive beams can be formed in parallel. These receive beams can be formed with broad transmit beams, to form an image of a subject region through a single transmit event. As a result, a very high imaging frame rate can be achieved and overall ultrasound session times can be reduced.

Reference is now made to the figures, where like components are designated by like reference numerals throughout the disclosure. Some of the infrastructure that can be used with embodiments disclosed herein is already available, such as general-purpose computers, computer programming tools and techniques, digital storage media, and communications networks. A computing device may include a processor such as a microprocessor, microcontroller, logic circuitry, or the like. The processor may include a special purpose processing device such as an ASIC, PAL, PLA, PLD, FPGA, or other customized or programmable device. The computing device may also include a computer-readable storage device such as non-volatile memory, static RAM, dynamic RAM, ROM, CD-ROM, disk, tape, magnetic, optical, flash memory, or other non-transitory computer-readable storage medium.

Various aspects of certain embodiments may be implemented using hardware, software, firmware, or a combination thereof. As used herein, a software module or component may include any type of computer instruction or computer executable code located within or on a computer-readable storage medium. A software module may, for instance, comprise one or more physical or logical blocks of computer instructions, which may be organized as a routine, program, object, component, data structure, etc., which performs one or more tasks or implements particular abstract data types.

In certain embodiments, a particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media. Some embodiments may be practiced in a distributed computing environment where tasks are performed by a remote processing device linked through a communications network.

The embodiments of the disclosure will be best understood by reference to the drawings. The components of the disclosed embodiments, as generally described and illustrated in the figures herein, could be arranged and designed in a wide variety of different configurations. Furthermore, the features, structures, and operations associated with one embodiment may be applicable to or combined with the features, structures, or operations described in conjunction with another embodiment. In other instances, well-known structures, materials, or operations are not shown or described in detail to avoid obscuring aspects of this disclosure.

Thus, the following detailed description of the embodiments of the systems and methods of the disclosure is not intended to limit the scope of the disclosure, as claimed, but is merely representative of possible embodiments. In addition, the steps of a method do not necessarily need to be executed in any specific order, or even sequentially, nor need the steps be executed only once.

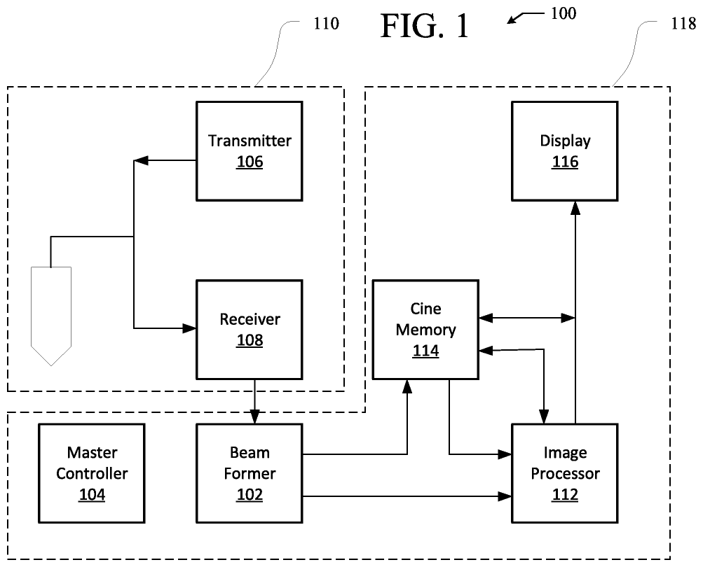

FIG. 1 is a schematic block diagram of one exemplary embodiment of a medical imaging device, such as an ultrasound imaging device 100. Those skilled in the art will recognize that the principles disclosed herein may be applied to a variety of medical imaging devices, including, without limitation, an X-ray imaging device, a computed tomography (CT) imaging device, a magnetic resonance imaging (MRI) device, and a positron-emission tomography (PET) imaging device. As such, the components of each device may vary from what is illustrated in FIG. 1.

In one embodiment, the ultrasound imaging device 100 may include an array focusing unit, referred to herein as a beam former 102, by which image formation can be performed on a scanline-by-scanline basis. The device may be controlled by a master controller 104, implemented by a microprocessor or the like, which accepts operator inputs through an operator interface and in turn controls the various subsystems of the device 100.

For each scanline, a transmitter 106 generates a radio-frequency (RF) excitation voltage pulse waveform and applies it with appropriate timing across a transmit aperture (defined, in one embodiment, by a sub-array of active elements) to generate a focused acoustic beam along the scanline.

RF echoes received by one or more receive apertures or receiver 108 are amplified, filtered, and then fed into the beam former 102, which may perform dynamic receive focusing, i.e., realignment of the RF signals that originate from the same locations along various scan lines. Collectively, the transmitter 106 and receiver 108 may be components of a transducer 110. Various types of transducers 110 are known in the ultrasound imaging art, such as linear probes, curvilinear probes, and phased array probes.

An image processor 112 may perform processing tasks specific to various active imaging mode(s) including 2D scan conversion that transforms the image data from an acoustic line grid into an X-Y pixel image for display. For other modes, such as a spectral Doppler mode, the image processor 112 may perform wall filtering followed by spectral analysis of Doppler-shifted signal samples using typically a sliding FFT-window. The image processor 112 may also generate a stereo audio signal output corresponding to forward and reverse flow signals. In cooperation with the master controller 104, the image processor 112 may also format images from two or more active imaging modes, including display annotation, graphics overlays and replay of cine loops and recorded timeline data.

A cine memory 114 provides resident digital image storage to enable single image or multiple image loop review, and acts as a buffer for transfer of images to digital archival devices, such as hard disk drives or optical storage. In some systems, the video images at the end of the data processing path may be stored to the cine memory. In state-of-the-art systems, amplitude-detected, beamformed data may also be stored in cine memory 114. For spectral Doppler mode, wall-filtered, baseband Doppler 1/Q data for a user-selected range gate may be stored in cine memory 114. Subsequently, a display 116, such as a computer monitor, may display ultrasound images created by the image processor 112 and/or images using data stored in the cine memory 114.

The beam former 102, the master controller 104, the image processor 112, the cine memory 114, and the display 116 can be included as part of a main processing console 118 of the ultrasound imaging device 100, which may include more or fewer components or subsystems than are illustrated. The ultrasound transducer 110 may be incorporated into an apparatus that is separate from the main processing console 118, e.g. in a separate apparatus that is wired or wirelessly connected to the main processing console 118. This allows for easier manipulation of the ultrasound transducer 110 when performing specific ultrasound procedures on a patient. Further, the transducer 110 can be an array transducer that includes an array of transmitting and receiving elements for transmitting and receiving ultrasound waves.

Those skilled in the art will recognize that a wide variety of ultrasound imaging devices are available on the market, and additional details relating to how images are generated is unnecessary for a thorough understanding of the principles disclosed herein. Specifically, the systems, methods, and computer-readable media described herein can be applied through an applicable ultrasound imaging device of the wide variety of ultrasound imaging devices available on the market.

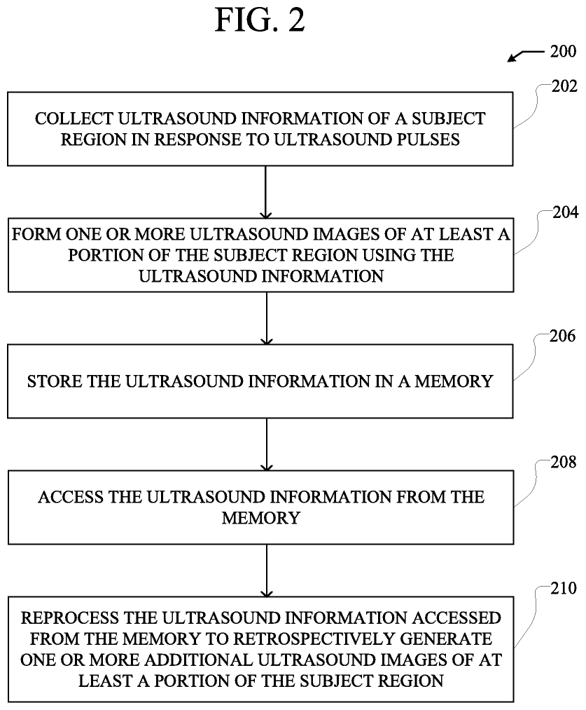

FIG. 2 is a flowchart 200 of an example method for storing and reprocessing collected ultrasound information to generate additional ultrasound image(s) after the ultrasound information is collected. The example method shown in FIG. 2, and other methods and techniques for ultrasound imaging described herein, can be performed by an applicable ultrasound imaging system, such as the ultrasound system 100 shown in FIG. 1. For example, the techniques for ultrasound imaging described herein can be implemented using either or both the ultrasound transducer 110 and the main processing console 118, e.g. the image processor 112, of the ultrasound system 100.

At step 202, ultrasound information of a subject region is collected in response to ultrasound pulses transmitted toward the subject region. The ultrasound information can include applicable information related to the transmission and reflection of ultrasound to and from the subject region. Specifically, the ultrasound information can include transmit profiles of the ultrasound pulses transmitted toward the subject region through one or more transmission events. Further, the ultrasound information can include reflectivity information in response to the ultrasound pulses transmitted towards the subject region. Reflectivity information includes applicable information used in generating ultrasound images of at least a portion of the subject region. Specifically, reflectivity information can include information of reflections of ultrasound pulses transmitted into the subject region, e.g. information of backscattered ultrasound pulses. In turn and as will be discussed in greater detail later, the information of the reflections can be used to generate ultrasound images through an applicable imaging/image formation technique.

Ultrasound information collected at step 202 can include channel domain data. Channel domain data, as used herein, includes data generated from each transducer element and from every transmit/receive cycle that is used to produce an ultrasound image. For example, in a 128-channel system that is using a single focus zone and sampling to a depth of 16 cm in a curved array format there might be around 192 transmit receive cycles. Channel domain data can include data that is used to generate an ultrasound image before any processing is done on the data. For example, channel domain data can include data that is generated by an ultrasound transducer before the data is pre-processed for beamforming, before beamforming actually occurs, and/or before the data is post-processed after beamforming to generate an ultrasound image.

At step 204, one or more ultrasound images of at least a portion of the subject region can be formed using the ultrasound information. The one or more ultrasound images can be formed using the ultrasound information as the ultrasound information is gathered during an ultrasound session. Specifically, the one or more ultrasound images can be formed as live images during a live imaging session as one or more ultrasound transducers remain operationally coupled to the subject region, e.g. during or immediately after transmission of the ultrasound pulses. More specifically, the one or more ultrasound images can be generated during the live imaging session as the ultrasound information is collected by processing the ultrasound information in real-time as the ultrasound information is collected. In turn, the one or more ultrasound images can be presented to an operator during the ultrasound session, e.g. in real-time during the ultrasound session.

The one or more images of the subject region formed at step 204 can be included as part of the ultrasound information collected at step 202. Accordingly, the step of forming the one or more images can be included as part of step 202 of collecting the ultrasound information of the subject region. As follows and as will be discussed in greater detail later, the one or more images of the subject region can be reprocessed, as part of reprocessing the collected ultrasound information, to retrospectively generate one or more additional ultrasound images of at least a portion of the subject region. For example, the one or more images formed at step 204 can later be modified, as part of reprocessing the ultrasound information, to retrospectively generate one or more additional ultrasound images of at least a portion of the subject region.

The ultrasound information can be collected at step 202 and the one or more ultrasound images can be formed at step 204 according to an applicable ultrasound imaging mode. For example, the ultrasound information can be collected and the one or more ultrasound images can be formed using the ultrasound information, at steps 202 and 204, according to a B-mode imaging mode. Specifically, the ultrasound information can be collected at step 202 and the one or more ultrasound images can be formed at step 204 according to a first ultrasound imaging mode. In turn and as will be discussed in greater detail later, the ultrasound information can be reprocessed later to retrospectively generate one or more additional ultrasound images according to a second ultrasound imaging mode. Further and as will be discussed in greater detail later, the first and second ultrasound imaging modes can be different ultrasound imaging modes.

The ultrasound information can be collected at step 202 and the one or more ultrasound images can be formed at step 204 through a high frame rate ultrasound imaging mode. A high frame rate ultrasound imaging mode can include an imaging mode or modified imaging mode for gathering ultrasound information and/or generating ultrasound images at a frame rate that is higher than a conventional ultrasound imaging mode. For example, a high frame rate ultrasound imaging mode can include a high frame rate B-mode imaging mode that acquires ultrasound information and/or generates ultrasound images at a higher frame rate than the conventional B-mode imaging mode. In another example, a high frame rate ultrasound imaging mode can achieve a frame rate of more than one hundred images per second.

A high frame rate ultrasound imaging mode can be achieved by transmitting a sequence of broad transmit beams toward the subject region. More specifically, the high frame rate ultrasound imaging mode can be achieved by transmitting the sequence of broad transmit beams toward the subject region without temporal gaps between the transmit beams. Further, the high frame rate ultrasound imaging mode can be achieved by transmitting a sequence of broad transmit beams towards the subject region over a plurality of different angles with respect to the subject region. For example, the broad transmit beams can be transmitted toward the subject region from different origins or across different steering angles to vary the angle that the broad transmit beams are transmitted toward the subject region. Transmitting the broad transmit beams towards the subject region over a plurality of different angles can facilitate retrospective focusing through the reprocessing of the gathered ultrasound information, as will be discussed in greater detail later.

Further, a high frame rate ultrasound imaging mode can be achieved through an applicable ultrasound scan format. For example, the high frame rate ultrasound imaging mode can be achieved through a linear scan format, a trapezoidal scan format, a vector scan format, a curved scan format, or a sector scan format. Additionally, the high frame rate ultrasound imaging mode can be applied in gathering ultrasound information as an applicable multi-dimensional array, e.g. a two dimensional array, a three dimensional array, and a temporal three dimensional array.

A high frame rate ultrasound imaging mode can be achieved through an applicable scan sequence for transmitting broad transmit beams towards the subject region. Specifically, the high frame rate ultrasound imaging mode can be achieved through an applicable scan sequence for transmitting broad transmit beams towards the subject region at varying angles with respect to the subject region.

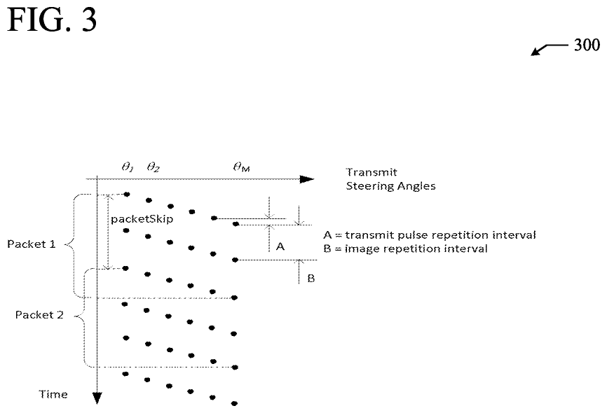

FIG. 3 illustrates an example scan sequence 300 of a high frame rate ultrasound imaging mode. As shown in the example scan sequence 300, the broad transmit beams at the varying angles θ₁, θ₂, . . . , θ_{M} can be transmitted sequentially. Specifically, if a total of M transmit angles are used for the broad transmit beams, then the broad transmit beams corresponding to these angles can be transmitted sequentially. In turn, after the corresponding broad transmit beams for all of the angles are transmitted, then the sequence can be repeated. The raw images formed for the different transmit angles can be summed coherently to improve resolution and the signal-to-noise ratio (SNR). The number of raw images summed at each pixel location can be fewer than M. The summed images can be evenly spaced in time, with the frame rate equal to PRF_tx/M, where PRF_tx is the transmit pulse repetition frequency. The packet size, or the number of image frames processed as a packet for flow or motion estimation, can be varied, as will be discussed in greater detail later, during retrospective processing in forming one or more additional images from the ultrasound information. The packetSkip, i.e. the number of frames skipped between neighboring packets, may be less than the packet size. As a result, there can be overlapping frames between neighboring packets.

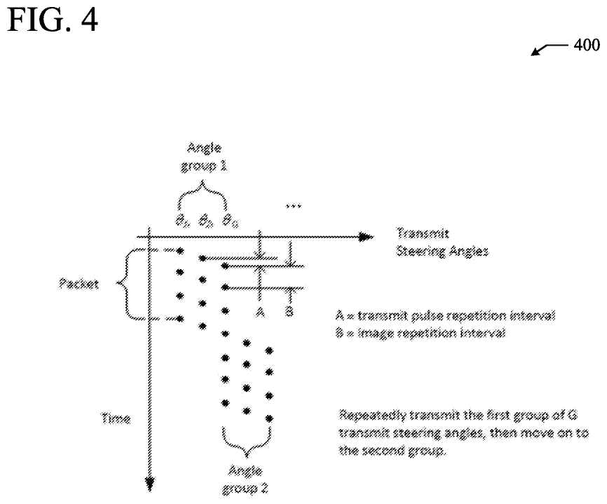

FIG. 4 illustrates another example scan sequence 400 of a high frame rate ultrasound imaging mode. In the scan sequence 400 shown in FIG. 4, a smaller number (G) of transmit angles form a group of angles, when compared to the scan sequence 300 shown in FIG. 3. This group is repeatedly scanned for N times before corresponding broad transmit pulses in another group of angles are transmitted towards the subject region. The angles of the different groups of angles can overlap. In the sequence 400 shown in FIG. 4, the frame rate achieved in the high frame rate ultrasound imaging mode is equal to PRF_tx/G. This is higher than the frame rate achieved through the scan sequence 300 shown in FIG. 3, as G is less than M.

While the transmit angles in the scan sequences 300 and 400 are shown as increasing monotonically within each cohort, as θ₁, θ₂, . . . , θ_{M}, the scan sequences 300 and 400 are not limited to monotonically increasing transmit angles. For example, the transmit angles can alternate as θ₁, θ_{M}, θ₂, θ_{M-1}, . . . . Alternatively, the transmit angles can be triangularly ordered. In turn, a scan sequence with triangularly ordered transmit angles can be used for tissue motion estimation and compensation.

With each transmit angle, it is understood that multiple firings may be employed to form a mini-sequence for extraction of non-linear information and improvement of the SNR. For example, a two firing mini-sequence can be transmitted through pulses of opposite signs or phases. In another example, a three firing mini-sequence can be transmitted by activating specific elements of an active transmit aperture while simultaneously inverting the pulse signs or phases.

The disclosure now describes an example technique for gathering the ultrasound information and forming the one or more ultrasound images based on the ultrasound information through a high frame rate B-mode imaging mode. Initially, the subject region is imaged in a conventional B-mode imaging mode, e.g. using focused transmit wavefronts, possibly with harmonic imaging, and spatial or frequency compounding. More specifically, the subject region can be imaged at a frame rate that is typically used in a conventional B-mode imaging mode, e.g. in the 10-100 Hz range.

Subsequently, a high frame rate B-mode imaging mode can be activated and the subject region can be imaged through the high frame rate B-mode imaging mode. The high frame rate B-mode imaging mode can be achieved through the previously described techniques for achieving a high frame rate imaging mode. For example, plane ultrasound waves can be launched at 10 kHz over various angles and each final image can be obtained by coherently summing the raw images from the various angles to achieve a frame rate of 10 kHz/10=1 kHz.

Switching to the high frame rate imaging mode, e.g. high frame rate B-mode imaging mode, can be controlled by an operator of the ultrasound system. Specifically, a user can start a setup mode for the high frame rate imaging mode. During the setup mode, the user can select a region of interest in the subject region and a desired frame rate for the high frame rate imaging mode, e.g. based on the velocity of blood flow or tissue motion. The region of interest can include the entire subject region or a portion of the subject region.

Once the high frame rate B-mode imaging mode is activated, the background B-mode image can be frozen. The background B-mode image can include all or a portion of the subjection region, potentially including the selected region of interest in the subject region. Then the region of interest is insonified repeatedly using broad beams, e.g. planar or spherical waves, of different incident angles, at the user-selected frame rate according to the high frame rate B-mode imaging mode.

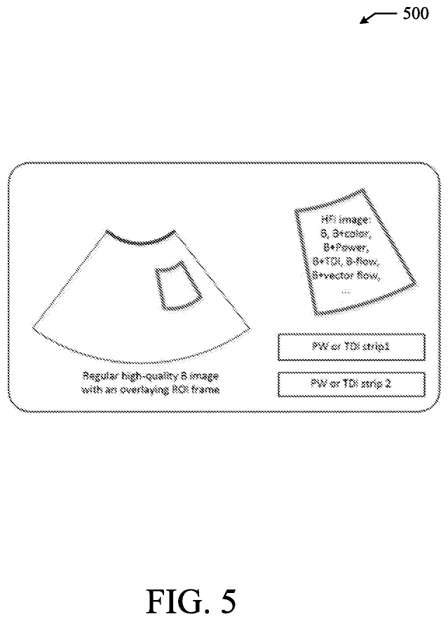

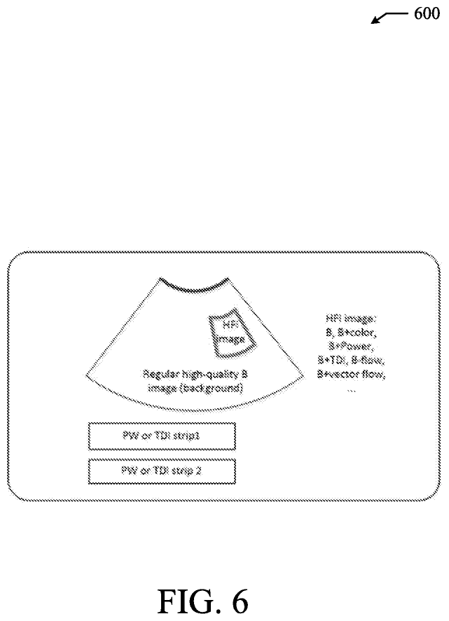

During the live imaging session, one or more images of the region of interest generated through the high frame rate imaging mode are displayed. The image(s) can be displayed in a separate region or embedded in the background B-mode image. FIG. 5 shows an example display format 500 where the high frame rate image(s) are displayed in a separate region from a background B-mode image during a live imaging session. In the example display format 500, the region of interest is shown in the background B-mode image. For example, a silhouette of the region of interest can be shown in the background B-mode image. FIG. 6 shows another example display format 600 where the high frame rate image(s) are displayed embedded in the background B-mode image.

A user can control imaging according to the high frame rate B-mode imaging mode during the live imaging session. Specifically, the user can adjust either or both the region of interest and the frame rate for the high frame rate B-mode imaging mode. In turn, either or both the previously defined settings for the high frame rate B-mode imaging mode and data collected through the high frame rate imaging mode can be removed from the memory. The user can also turn off imaging according to the high frame rate B-mode imaging mode during the live imaging session to convert back to the conventional imaging mode, e.g. B-mode imaging mode.

Returning back to the flowchart 200 shown in FIG. 2, at step 206 the collected ultrasound information is stored in a memory. The ultrasound information can include collected raw image data, e.g. at step 202. For example, the ultrasound information can include channel domain data gathered during a high frame imaging mode, e.g. a high frame rate B-mode imaging mode. Further, the ultrasound information can include image data of one or more generated ultrasound images, e.g. the ultrasound images generated at step 204. For example, the ultrasound information can include image data of the one or more images generated during a high frame imaging mode, e.g. a high frame rate B-mode imaging mode. The ultrasound information can be stored in the memory in an applicable format. Specifically, channel domain data included in the ultrasound information can be stored in an RF data format. Further, image data included in the ultrasound information can be stored in an IQ data format.

The ultrasound information stored in the memory at step 206 can be collected and/or generated, e.g. at steps 202 and 204, over a specific amount of time. Specifically, when the subject region is a patient, the ultrasound information can be collected and generated over at least one cardiac cycle of the patient to create at least one cardiac cycle of ultrasound information. Further, the ultrasound information can be collected and generated over one or more seconds of time to create one or more seconds of ultrasound information.

The memory can be a cyclical memory in which data can be deleted from the memory as new data is added to the memory, e.g. on a per-storage amount basis. For example, the image data, e.g. IQ image data after summation, can be stored in a cine memory in a cyclical fashion. The image data can be stored based on storage requirements for the data. Further in the example, if the IQ image contains 4e4 samples (200×200), and each sample is 8 bytes, at a frame rate of 1e3 Hz, to store 10 seconds of data, 4e4 samples/frame*8 bytes/sample*1 e3 frames/sec*10 seconds=3.2 GB is needed to store the image data.

The ultrasound information can be stored in the memory through a cyclical technique during a live ultrasound session. Specifically, ultrasound information generated through the live ultrasound session can be continuously added to the memory in the order that it is created or otherwise collected during the live ultrasound session. In turn, if the memory becomes full during the live ultrasound session, then the oldest ultrasound information can be deleted from the memory while the newest generated ultrasound information is added to the memory.

At step 208 the ultrasound information is accessed from the memory. At step 210, the ultrasound information that is accessed from the memory is reprocessed to retrospectively generate one or more additional ultrasound images of at least a portion of the subject region. Retrospectively generating the one or more additional ultrasound images, as used herein, includes generating the one or more additional ultrasound images at an applicable time after the ultrasound information is created. Specifically, retrospectively generating the one or more additional ultrasound images can include generating the one or more additional ultrasound images after an ultrasound session in which the ultrasound information is collected has ended. For example, the one or more additional ultrasound images can be retrospectively generated by reprocessing the ultrasound information after the subject region is no longer operationally coupled to one or more ultrasound transducers of an ultrasound system. In another example, the one or more additional ultrasound images can be retrospectively generated by reprocessing the ultrasound information after an ultrasound examination of a patient has ended.

When the ultrasound information includes image data of the one or more ultrasound images formed at step 204, the image data can be reprocessed to generate the one or more additional ultrasound images. Specifically, image data stored in an IQ data format in the memory can be reprocessed to generate the one or more additional ultrasound images. For example, the one or more images, stored in an IQ data format, can be modified to generate the one or more additional ultrasound images through reprocessing of the ultrasound information. Storing and reprocessing image data, when compared to storing and reprocessing channel data, is advantageous as less storage space and computational power is needed to store and reprocess the image data.

When the ultrasound information includes collected channel domain data, e.g. the channel domain data collected at step 202, the channel domain data can be reprocessed to generate the one or more additional ultrasound images. Specifically, channel domain data stored in an RF data format in the memory can be reprocessed to generate the one or more additional ultrasound images. In reprocessing the collected channel domain data to generate the one or more additional ultrasound images, the collected channel domain data can be reprocessed according to values of one or more image formation parameters. Image formation parameters include applicable parameters that can be varied in applying an applicable ultrasound imaging mode to generate ultrasound images from channel domain data. For example image formation parameters can include one or a combination of a receive aperture size parameter, an apodization parameter, and a distribution of sound speed parameter. Reprocessing collected channel domain data, when compared to reprocessing image data, is advantageous in that it provides greater flexibility in image formation, e.g. by providing the ability to adjust image formation parameters.

The applied image formation parameters can be selected by a user. Specifically, a user can select an ultrasound imaging mode for generating the one or more additional ultrasound images, thereby effectively selecting the image formation parameters that are specific to the selected ultrasound imaging mode. Further, the values of the image formation parameters can be selected, e.g. by a user. Specifically, the values of the image formation parameters can be selected, e.g. by a user, after the ultrasound information is gathered or otherwise created. More specifically, the values of the image formation parameters can be selected as part of reprocessing the ultrasound information to retrospectively generate the one or more additional ultrasound images.

The ultrasound information can be reprocessed according to an applicable ultrasound imaging mode. Specifically, the ultrasound information can be reprocessed according to a second ultrasound imaging mode in comparison to the first ultrasound applied in generating the one or more ultrasound images at step 204. The second ultrasound imaging mode applied in generating the one or more additional ultrasound images can be different from the first ultrasound imaging mode applied in generating the one or more ultrasound images at step 204. The second ultrasound imaging mode can be an applicable ultrasound imaging mode for generating ultrasound images. For example, the second ultrasound imaging mode can include a B-mode imaging mode, a color-flow mode, a pulse-wave Doppler mode, a tissue Doppler mode, a B-flow mode, a tissue strain mode, a tissue elasticity mode, and a vector flow mode.

The ultrasound imaging mode to apply in reprocessing the ultrasound information can be selected, e.g. after the ultrasound information is stored in the memory. Specifically, a user can select the ultrasound imaging mode to apply in reprocessing the ultrasound information to retrospectively generate the one or more additional ultrasound images. In turn, a cine loop of the one or more additional ultrasound images can be played back at a specific speed. Specifically, a user can select a playback speed for the cine loop of the one or more additional ultrasound images and the additional ultrasound images can be played back in the cine loop according to the selected playback speed. Additionally and as part of reprocessing the ultrasound information, pulse wave (PW) or Tissue Doppler imaging (TDI) cursors can be placed, e.g. by a user, in the region of interest, e.g. the additional ultrasound image(s) of the region of interest, to inspect blood flow or tissue motion.

The following description includes examples of ultrasound imaging modes and combinations of ultrasound imaging mode that can be applied in reprocessing the ultrasound information. Combination imaging modes, as used herein, can include two ultrasound imaging modes, that are potentially applied at different times. For example, combination imaging modes can include a first imaging mode that is applied during a live ultrasound imaging session and a second imaging mode that is applied to retrospectively generate one or more additional ultrasound images, e.g. after the live ultrasound imaging session has ended.

In a first example, a B+Color+PW combination imaging mode is applied. In this combination imaging mode, color flow images in a region of interest are displayed at a user-selected frame rate. Further in this combination imaging mode, a background B-mode image within the region of interest is derived from one or more images gathered through a high frame rate imaging mode, herein referred to as high frame rate images. The high frame rate images can be created with temporal averaging to improve the SNR. For a given packet size, a higher frame rate can lead to more data overlap when producing successive output color frames. Alternatively, the packet size can be adjusted with the frame rate to balance between flow dynamics and flow sensitivity. In this combination imaging mode, PW strips at multiple user-selected locations can be computed and displayed.

In a second example, a B+TDI+TDI strip combination imaging mode is applied. In this combination imaging mode, TDI images in a region of interest is displayed at a user-selected frame rate. Further in this combination imaging mode, a background B-mode image within the region of interest is derived from one or more high frame rate images. The high frame rate images can be created with temporal averaging to improve the SNR. In this combination imaging mode, TDI strips of multiple user-selected locations can be computed and displayed.

In a third example, a B+B-flow combination imaging mode is applied. The B-flow images can be generated at a high frame rate of a high frame rate imaging mode, which can reach thousands of frames per second. Since human eyes can only perceive a much lower rate, such as 30 Hz, inter-frame low-pass filtering and decimation can be applied to display the images in real-time. During review, the B-flow images can be played back in slow-motion without temporal decimation, so that the detailed flow dynamics can be visualized.

In a fourth example, a B+Shear Wave Elastography (SWE)+Color combination imaging mode is applied. In the combination imaging mode, plane or diverging waves are used to detect shear waves caused by external vibration or generated by acoustic radiation force. B-mode images are acquired at frame rates of several thousands of Hz for the detection of tissue motion. With filtering to remove tissue signal, the image blood flow in tissue can be displayed using the same set of data.

In a fifth example, a B+Vector Flow combination imaging mode is applied. IN the combination imaging mode, vector flow images can be generated using speckle tracking on high frame rate B-mode images after clutter filtering. Alternatively, Doppler shifts estimated from different transmit/receive angles can be solved to obtain true flow velocity and direction.

The one or more additional ultrasound images can be displayed through an applicable display format. Specifically, the images created through the previously described combination imaging modes can be displayed through an applicable display format. For example, the display formats 500 and 600 shown in FIGS. 5 and 6 can be utilized in displaying the images created through the previously described combination imaging modes.

The technology described herein has many potential clinical applications. For example, the technology described herein can be applied in providing transcranial color Doppler analysis, which provides better sensitivity than conventional color flow analysis. Further, the technology described herein can be applied in providing Cardiac tissue motion analysis, which has higher spatial and temporal resolution than conventional TDI. Additionally, the technology described herein can be applied in providing visualization of flow dynamics in the presence of plaque. Further, the technology described herein can be applied in providing synchronized multi-gate Doppler strips of blood flow or tissue, e.g. peak arrival times at different locations. Additionally, the technology described herein can be applied in providing visualization of cardiac valves of a fetal heart.

This disclosure has been made with reference to various exemplary embodiments including the best mode. However, those skilled in the art will recognize that changes and modifications may be made to the exemplary embodiments without departing from the scope of the present disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the following claims.

What is claimed is:

1. A method for performing ultrasound imaging comprising:
collecting ultrasound information of a subject region in response to ultrasound pulses transmitted toward the subject region;
forming one or more ultrasound images of at least a portion of the subject region using the ultrasound information;
storing the ultrasound information in a memory;
accessing the ultrasound information from the memory; and
reprocessing the ultrasound information accessed from the memory to retrospectively generate one or more additional ultrasound images of at least a portion of the subject region.

2. The method of claim 1, wherein the one or more ultrasound images are generated as the ultrasound information is collected by processing the ultrasound information in real-time as the ultrasound information is collected.

3. The method of claim 1, wherein the ultrasound information is gathered during a session of transmitting the ultrasound pulses toward the subject region and the ultrasound information is reprocessed to retrospectively generate the one or more additional ultrasound images after the session.

4. The method of claim 1, wherein the ultrasound information includes either or both channel domain data and image data of the subject region.

5. The method of claim 4, further comprising storing the ultrasound information in the memory in either or both a radio frequency (RF) data format or an in-phase quadrature (IQ) data format corresponding to the channel domain data and the image data of the subject region.

6. The method of claim 1, wherein the ultrasound information includes channel domain data, the method further comprising:
storing the channel domain data in a radio frequency (RF) data format in the memory; and
reprocessing the channel domain data accessed from the memory according to values of one or more image formation parameters to retrospectively generate the one or more additional ultrasound images.

7. The method of claim 6, wherein the values of the one or more image formation parameters are selected after the ultrasound information is gathered and the one or more ultrasound images are formed from the ultrasound information.

8. The method of claim 7, wherein the values of the one or more image formation parameters are selected by a user.

9. The method of claim 6, wherein the one or more image formation parameters include one or a combination of a receive aperture size parameter, one or more apodization parameters, and a distribution of sound speed parameter.

10. The method of claim 1, further comprising:
including image data of the one or more ultrasound images in the ultrasound information;
storing the ultrasound information including the image data of the one or more ultrasound images in the memory; and
reprocessing the image data of the one or more ultrasound images to retrospectively generate the one or more additional ultrasound images based on the one or more ultrasound images.

11. The method of claim 10, further comprising modifying the one or more ultrasound images to retrospectively generate the one or more additional ultrasound images based on the one or more ultrasound images.

12. The method of claim 1, wherein the one or more ultrasound images are generated through a first ultrasound imaging mode and the one or more additional ultrasound images are generated through a second ultrasound imaging mode.

13. The method of claim 12, wherein the first ultrasound imaging mode and the second ultrasound imaging mode are different ultrasound imaging modes.

14. The method of claim 13, wherein the first ultrasound imaging mode is a high frame rate B-mode.

15. The method of claim 14, wherein the high frame rate B-mode is achieved by transmitting a sequence of broad transmit beams as part of the ultrasound pulses transmitted toward the subject region.

16. The method of claim 15, wherein the broad transmit beams are transmitted toward the subject region at varying angles with respect to the subject region.

17. The method of claim 13, wherein the second ultrasound imaging mode includes at least one of B-mode, color-flow mode, pulse-wave Doppler mode, tissue Doppler mode, B-flow mode, tissue strain mode, tissue elasticity mode, and vector flow mode.

18. The method of claim 1, wherein the subject region is a portion of a patient and the memory is cyclical memory, further wherein the ultrasound information is stored over a period of a plurality of cardiac cycles of the patient in a cyclical manner in the cyclical memory.

19. A system for performing ultrasound imaging comprising:
an ultrasound transducer configured to:
collect ultrasound information of a subject region in response to ultrasound pulses transmitted toward the subject region;
a main processing console configured to:
form one or more ultrasound images of at least a portion of the subject region using the ultrasound information;
store the ultrasound information in a memory;
access the ultrasound information from the memory; and
reprocess the ultrasound information accessed from the memory to retrospectively generate one or more additional ultrasound images of at least a portion of the subject region.

20. A system for performing ultrasound imaging comprising:
one or more processors; and
a computer-readable medium providing instructions accessible to the one or more processors to cause the one or more processors to perform operations comprising:
collecting ultrasound information of a subject region in response to ultrasound pulses transmitted toward the subject region;
forming one or more ultrasound images of at least a portion of the subject region using the ultrasound information;
storing the ultrasound information in a memory;
accessing the ultrasound information from the memory; and
reprocessing the ultrasound information accessed from the memory to retrospectively generate one or more additional ultrasound images of at least a portion of the subject region.
